(19) United States
(12) Patent Application Publication
(10) Pub. No.: US 2015/0228205 A1
(43) Pub. Date: Aug. 13, 2015

(54) COVERING FOR RESUSCITATION AND A METHOD FOR MANUFACTURING THEREOF

(71) Applicant: AID ONE SOLUTIONS OY, Espoo (FI)

(72) Inventors: Jussi Koskimaki, Espoo (FI); Jarkko Valtanen, Helsinki (FI)

(21) Appl. No.: 14/430,784

(22) PCT Filed: Sep. 24, 2013
(86) PCT No.: PCT/FI2013/050921

§ 371 Date: Mar. 24, 2015

(30) Foreign Application Priority Data
Sep. 24, 2012 (DE) 10 2012 217 213.0

(51) Int. Cl. G09B 23/28; A61F 7/00; A61B 5/0402; A61B 5/0205; A61M 16/04; A61B 5/00

(52) U.S. Cl. CPC G09B 23/288; A61M 16/049; A61B 5/6892; A61B 5/0402; A61B 5/0006; A61B 5/0008; A61B 5/02055; A61B 5/7405; A61B 5/742; A61B 5/486; A61F 7/0097

(57) ABSTRACT

A resuscitation covering (11, 41), intended for cardiac massage, includes a compression area (21) and is a part of an emergency blanket (10) and is removable from the rest of the emergency blanket (10). A method for manufacturing a resuscitation covering (11) is also described.

The invention relates to emergency first aid, and specifically to accessories useful in the resuscitation of unconscious persons.

### PRIOR ART

People are often reluctant to help victims in situations that demand first aid. Many lack confidence in their first aid skills.

What happens as a result is that sometimes in an accident or sudden illness situation people remain waiting for the arrival of professional assistance at the scene of resuscitation instead of starting resuscitation on their own initiative.

For a person demanding resuscitation, every delayed minute before the start of resuscitation can be critical from the standpoint of survival or deterioration of injuries. After all, in the incident of cardiac arrest, for example, the risk of brain damage increases rapidly.

### SUMMARY

One objective of the invention is to eliminate or at least reduce the prior art drawbacks. It is an object of the invention to lower the threshold for bystanders to commence resuscitation on their own initiative, thus enabling an earlier start to the resuscitation of an unconscious person.

Some objectives of the invention are accomplished with a resuscitation covering presented in claim 1 and in the parallel claim 13, as well as with a resuscitation covering manufacturing method presented in claim 13.

The resuscitation covering according to the invention is characterized by what is presented in claim 1 directed to a resuscitation covering.

Preferred embodiments for the resuscitation covering and the method are set forth in dependent claims.

### BENEFITS OF THE INVENTION

The resuscitation covering according to the invention for cardiac massage comprises a compression area and makes up part of an emergency blanket from which the resuscitation covering is removable. By virtue of the compression area, the commencement of cardiac massage at a correct location can be successfully and quickly managed even by a person with hardly any first aid skills.

The emergency blanket is most preferably an emergency blanket the size of 2,1 m×1,6 m consistent with any of the DIN standards 13157, 13169, 13164, 14142 or 13160. In some countries, for example in Germany, the emergency blanket is a statutory item in a first aid kit obligatory in every passenger car and in every workplace (incl. schools).

The resuscitation covering set up as a part of this type of emergency blanket is accessible as readily as possible with regard to most resuscitation situations.

Even a person who has passed a first air course can be insecure in the resuscitation situation, if not sure about the level of his/her skills. The combination of a resuscitation covering of the invention and an emergency blanket endeavors to make critical information more readily available for the rescuer in a resuscitation situation. Thereby is also provided a visual model applicable for resuscitation, which the emergency center duty officer can use as a guide when instructing a resuscitator or a first aid giver.

For a person trained in first aid, the combination resuscitation covering and emergency blanket provides a quick refresher of his/her skills, thereby enhancing the helper's self-confidence to perform in a resuscitation situation.

When a resuscitation covering is integrated with an emergency blanket in such a way that between the resuscitation covering and the emergency blanket lies at least one predetermined breaking point or line, the resuscitation covering is removable from the emergency blanket without a cutting tool.

When a resuscitation covering further includes a mouth-to-mouth resuscitation area with an existing or to-be-formed opening, the resuscitation covering not only lowers the threshold for cardiac massage but also for administering the actual cardiopulmonary resuscitation or mouth-to-mouth resuscitation. It is for hygienic reasons that many people find it uncomfortable to administer mouth-to-mouth resuscitation. The resuscitation covering may deter the secretions or bodily fluids of a resuscitated person from ending up in or on the rescuer's mouth, hands or clothes.

When a mouth-to-mouth resuscitation area includes at least one predetermined breaking point for a valve or a blowpipe, it is possible to break the mouth-to-mouth resuscitation area without a cutting tool. This eliminates the need of looking for a cutting tool in a resuscitation situation.

The mouth-to-mouth resuscitation area can be provided with a preexisting valve or blowpipe. Hence, the resuscitation covering is more quickly deployable.

The resuscitation covering may also comprise a recess for being placed against the neck of a resuscitated person. This facilitates making sure that the resuscitation covering has its resuscitation area set in a correct location, whereby the cardiac massage can be expected to be more effective. The heart in most people sits slightly to the left underneath the sternum (more specifically, see http://de.wikipedia.org/wiki/Herz).

When a resuscitation covering is at least partly transparent, the resuscitation covering is applicable for example in an accident situation for covering a person to be assisted.

When a resuscitation covering is at least partly transparent, it will be easier to confirm a correct alignment of the resuscitation covering to match the anatomy of a resuscitated person.

The method of making preparations for resuscitating a person comprises the detachment of a resuscitation covering according to a first aspect of the invention from an emergency blanket and its placement on top of the thorax of a person about to receive resuscitation.

When the predetermined breaking point of a resuscitation covering is ruptured and the resulting hole is fitted with a valve or a blowpipe, the resuscitation covering shall be ready for service also in mouth-to-mouth resuscitation.

When the recess present in a resuscitation covering is placed against the neck of a resuscitated person, the resuscitation covering shall place its compression area more likely in alignment with the resuscitated person's heart for a speedier commencement of effective resuscitation.

The resuscitation covering intended for cardiac massage according to a parallel embodiment of the invention comprises a compression area and a mouth-to-mouth resuscitation area with an existing or to-be-formed opening.

Thus, according to a first preferred aspect, the mouth-to-mouth resuscitation area includes at least one predetermined breaking point for an opening to be made for a valve or a blowpipe. According to a second preferred aspect, the mouth-to-mouth resuscitation area has an opening therein pre-fitted with a valve or a blowpipe.

### DESCRIPTION OF THE FIGURES

The resuscitation covering and the method according to the invention will now be described more closely by way of exemplary embodiments depicted in the accompanying drawings. In the drawings:

FIG. 1 shows one exemplary resuscitation covering in attachment with an emergency blanket according to one preferred embodiment of the invention,

FIG. 2 shows one exemplary resuscitation covering according to one preferred embodiment of the invention,

FIG. 3 shows one exemplary blowpipe installation procedure on a resuscitation covering's mouth-to-mouth resuscitation area according to one preferred embodiment of the invention,

FIG. 4 shows one exemplary resuscitation covering provided with a neck recess according to one preferred embodiment of the invention, and

FIG. 5 shows one exemplary folded-up emergency blanket according to one preferred embodiment of the invention.

### DETAILED DESCRIPTION OF THE FIGURES

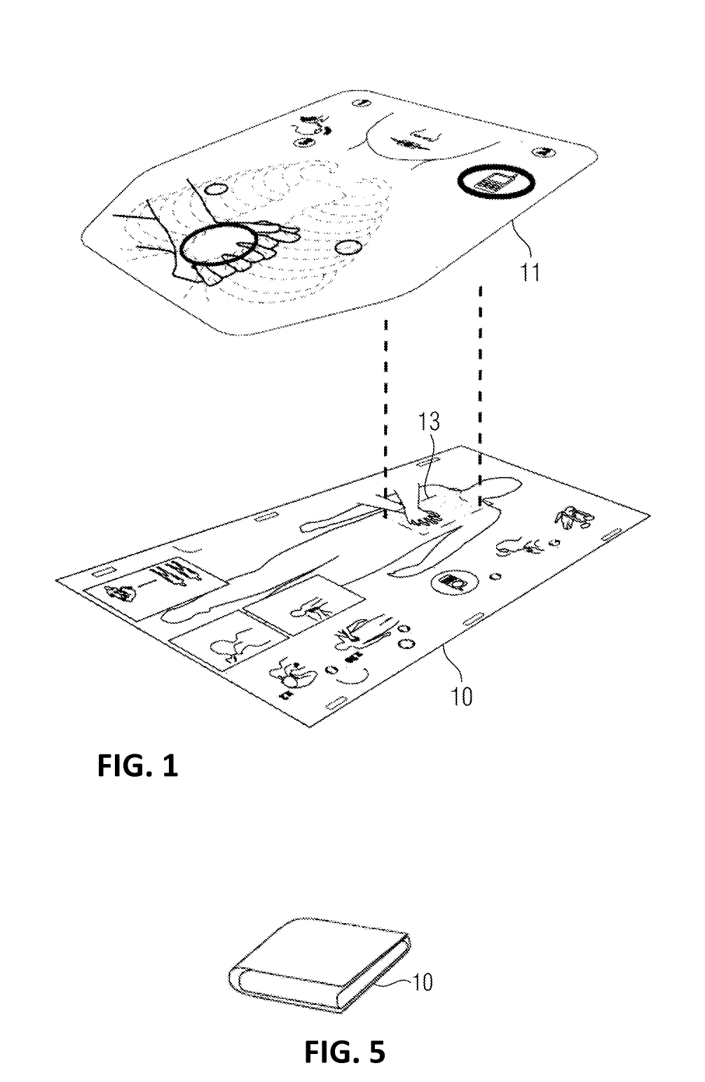

FIG. 1 shows an emergency blanket 10, wherein a translucent or opaque plastic, fabric or other material is printed with an instruction set containing instructions and alignment aids for the administration of approved first aid and cardiac massage. According to a preferred embodiment, the emergency blanket 10 is an emergency blanket the size of 2,1 m×1,6 m, which is consistent with DIN standard 13157, 13169, 13164, 14142 or 13160 and which is provided with printing.

According to the invention, the emergency blanket 10 comprises a resuscitation covering 11, which is intended for cardiac massage and comprises a compression area 21 and is a part of the emergency blanket 10. The resuscitation covering 11 is detachable from the emergency blanket 10, most preferably at a predetermined breaking point 13.

The emergency blanket 10 and/or the resuscitation covering 11 can be made from a membrane or fabric, making either or both foldable into a compact condition and thereby as a separate item easy to take along and readily disposable in a first aid kit or case (FIG. 5). According to one example, the emergency blanket 10 and the resuscitation covering 11 are made of metalized polyethylene terephthalate MPET.

The structure can also be a double layer structure. According to one example regarding the use of a double layer structure, one employed layer comprises, according to the prior art, most preferably a thin (for example 12 μm thin), tear-proof, transparent and waterproof polyester film (PET) and the other layer comprises an extremely thin, lightly transparent (for example 1%) aluminum layer.

When using a triple layer structure, it is possible to employ a metal/PET/metal or PET/metal/PET layering as disclosed in the prior art.

The resuscitation covering 11 can be furnished with an attachment or attachments (which can also be mutually dissimilar) facilitating its retention on a person receiving resuscitation. A possibly employed attachment comprises one or more adhesive films, rubber bands or other fastening tapes.

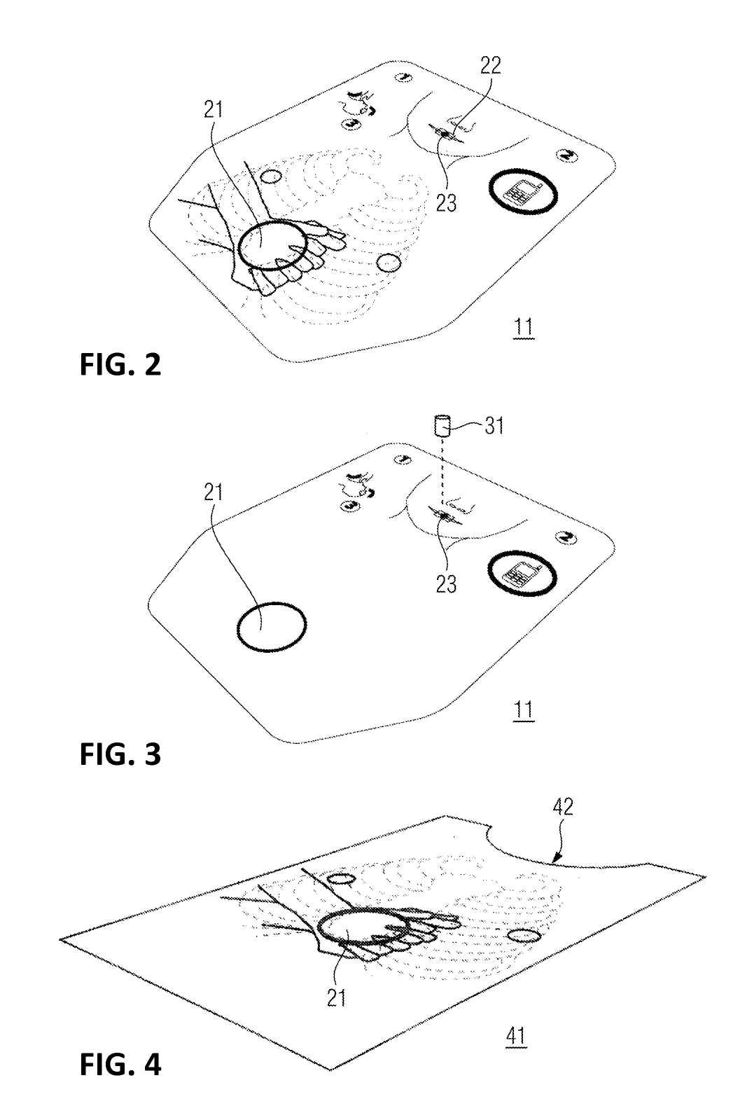

A resuscitation covering 11 shown in the embodiment of FIG. 2 is provided with alignment markers to assist in aligning a compression area 21 of the resuscitation covering 11, such that the compression area 21 is placed on top of the cardiac massage position.

When the employed alignment markers are
i) markers for top and bottom ends of sternum, and/or
ii) markers for positions of nipples;

the resuscitation covering 11 can be aligned by means of the alignment markers with a desired location. The resuscitation covering 11 may include either both or just one of the alignment markers.

The resuscitation covering 11 can be provided permanently with a mouth-to-mouth resuscitation area 22. The mouth-to-mouth resuscitation area 22 may include an opening and can be provided with an artificial respiration mouthpiece (i.e. a blowpipe 31, cf. FIG, 3) or a valve installed in the manufacturing process. Optionally, the mouth-to-mouth resuscitation area 22 may include a predetermined breaking point 23 without the valve or blowpipe 31.

The mouth-to-mouth resuscitation area 22 can be used as an artificial respiration shield.

As shown in FIG. 4, a resuscitation covering 41 according to a second embodiment of the resuscitation covering can be aligned by means of a neck recess 42 in compliance with the neck of a resuscitated person.

Alternatively, the resuscitation covering 41 can have its top portion designed in such a way that the neck recess 42 is identical in shape to the one in a bib, whereby the resuscitation covering 41 fits against the neck of a patient.

The resuscitation covering 11, 41 of the invention and/or the emergency blanket 10 have also most preferably instructions printed thereon for the administration of approved cardiac massage and other first aid.

The resuscitation covering 11, 41 can be supplemented with various electronic and/or mechanical accessories assisting or facilitating the resuscitation effort, for example with devices indicating a proper compression force and compression frequency by means of sound, light, or a counterforce.

The resuscitation covering 11, 41 or the emergency blanket 10 can be provided with a simple Bluetooth communication instrument (or the like short-range radio link), whereby the rescuer's mobile phone or other communication instrument can be set to function with a speaker or other user interface, for example a display, present in the resuscitation covering 11, 41 or in the emergency blanket 10, thus providing real time instructions from professionals.

The resuscitation covering 11, 41 can be furnished with mechanical and/or electronic accessories facilitating the work of professional rescuers and improving patient safety. Such accessories include, among others, sensors for measuring oxygen saturation, core temperature, heart rate, blood pressure, movement.

According to one example, the sensors for measuring vital functions (e.g. heart rate, temperature, ECG) of a patient can be integrated, according to one embodiment of the invention, with the resuscitation covering 11, 41 at such locations that, when using the resuscitation covering, these sensors settle in correct positions on a patient's body for example for measuring the above-mentioned parameters reflecting the vital functions of the patient. The sensors can be permanently installed in a resuscitation covering, or can be removable and comprise an adhesive surface covered by a protective sticker, said adhesive surface enabling an attachment of the sensor to the body of a patient.

The sensors can be wired to a resuscitation covering, in which case the covering preferably comprises wirings required by the sensors and an interface for connecting with an external device, for example with a professional grade monitoring device. According to one example, the sensors may also comprise wireless data transfer instruments for transmitting measurement data for example to a measurement application of the user's communication device, such as a mobile communicator. The measurement application, on the other hand, can deliver the measurement data of the sensors, as such or after being processed by the application, further for example to rescue services. According to one example, the rescue services can supply information, for example resuscitation instructions and the like, to the application and further to be presented to a user either by way of the user's communicator and/or by way of a user interface present in the resuscitation covering.

In the resuscitation covering (most preferably a thin film product) 11, 41, the wirings required by various measurement devices can be connected by printing and, if necessary, the wirings of several resuscitation coverings 11, 41 can be extended collectively to one place or terminal.

The resuscitation covering 11, 41 can also have heating or cooling associated therewith. According to one example, heating can be implemented by means of electric heating resistors integrated with a resuscitation covering. According to one example, the resuscitation covering or at least a part thereof comes in the form of a layered structure, wherein at least one section comprises a cold energy storage structure, a braiding or gel. The structure may also include subsections, which contain chemical substances, and which are separated with breakable or otherwise removable intermediate sections in such a way that the separation of intermediate sections brings the chemical substances together, resulting in a chemical reaction and, depending on the substances, chemical cooling or heating. It should be noted that the resuscitation covering may comprise subsections with several different chemical substances, such that first sections serve for cooling and second sections for heating.

The resuscitation covering 11 of FIG. 3 can also be manufactured and sold as a separate article, i.e. without an emergency blanket 10. Hence, the resuscitation covering 11 is available separately and a detachment from the emergency blanket 10 is not necessary for putting the resuscitation covering 11 to use.

the invention is further related to the manufacture of a resuscitation covering described in this document and/or an emergency blanket comprising the same, as well as for the implementation of features included therein, such as for the implementation of predetermined breaking points (13) or lines, a mouth-to-mouth resuscitation area formed or to be formed with an opening, a recess, a wiring. a sensor network which is wired or based on wireless data transfer, user interface instruments and/or sections and systems applicable for heating or cooling.

The foregoing description presents just a few embodiments for a solution of the invention. The principle according to the invention can naturally be diversified within the scope of protection defined by the claims, regarding for example implementation details and fields of use. The invention is not to be construed in the sense of being limited just to the appended claims, but it should be appreciated to encompass all legal equivalents thereof and combinations of the described embodiments.

1. A resuscitation covering (11, 41), characterized in that it is the resuscitation covering (11, 41), which is intended for cardiac massage and comprises a compression area (21) and is a part of an emergency blanket (10) and is removable from the rest of the emergency blanket (10).

2. A resuscitation covering (11, 41) as set forth in claim 1, which is integrated with an emergency blanket (10) in such a way that between the resuscitation covering (11, 41) and the emergency blanket (10) lies at least one predetermined breaking point (13) or line.

3. A resuscitation covering (11) as set forth in claim 1, which further includes a mouth-to-mouth resuscitation area (22) formed or to be formed with an opening.

4. A resuscitation covering (11) as set forth in claim 3, wherein the mouth-to-mouth resuscitation area (22) includes at least one predetermined breaking point (23) for a valve or blowpipe (31), or is fitted with a valve or blowpipe (31).

5. A resuscitation covering (41) as set forth in claim 1, which includes a recess (42) for placing the same against the neck of a resuscitated person.

6. A resuscitation covering (11, 41) as set forth in claim 1, wherein the emergency blanket (10) and/or the resuscitation covering (11) are/is at least partly transparent.

7. A resuscitation covering (11, 41) as set forth in claim 1, wherein the emergency blanket (10) and/or the resuscitation covering (11) comprise/comprises a sensor which is wired or based on wireless data transfer for the measurement of a person's vital function, for example heart rate, temperature, or ECG.

8. A resuscitation covering (11, 41) as set forth in claim 7, wherein the sensor is placed in the resuscitation covering at a location which, when deploying the resuscitation covering, is anatomically aligned with the point of measurement for a parameter being measured with this particular sensor.

9. A resuscitation covering (11, 41) as set forth in claim 7, wherein the sensor is removable from the resuscitation covering and comprises an adhesive surface for attachment to a person's body.

10. A resuscitation covering (11, 41) as set forth in claim 1, wherein the resuscitation covering (11) and/or the emergency blanket (10) comprise/comprises a wiring for sensors, and/or the resuscitation covering and/or the sensors comprise data transfer instruments for conveying measurement data to an external data processing instrument, such as a mobile communicator.

11. A resuscitation covering (11, 41) as set forth in claim 1, wherein the resuscitation covering (11) and/or the emergency blanket (10) comprise/comprises user interface instruments, for example sound producing instruments or a display, for presenting the user with resuscitation instructions or rescue service messages preferably delivered by way of a wireless data transfer path.

12. A resuscitation covering (11, 41) as set forth in claim 1, wherein the resuscitation covering (11) and/or the emergency blanket (10) comprise/comprises a section for heating or a section for cooling, and wherein the heating or cooling is carried out electrically or with layer structures connectably separated from each other, the layer structures comprising chemical substances, such that the result of combining the sections and the chemical substances with each is the generation of either a cooling or heating effect in the resuscitation covering (11) and/or in the emergency blanket (10).

13. A method for manufacturing a resuscitation covering (11, 41) as set forth in claim 1, characterized in that the method comprises producing the resuscitation covering (11, 41), which is intended for cardiac massage, and which is provided with a compression area (21) in such a way that it is a part of an emergency blanket (10) and is removable from the rest of the emergency blanket (10).

14. A resuscitation covering (11), characterized in that the resuscitation covering (11), which is intended for cardiac massage, comprises a compression area (21) and a mouth-to-mouth resuscitation area (22) formed or to be formed with an opening.

15. A resuscitation covering (11) as set forth in claim 14, wherein:
the mouth-to-mouth resuscitation area (22) is provided with at least one predetermined breaking point (23) for making a hole for a valve or blowpipe (31); or
the mouth-to-mouth resuscitation area (22) has a hole therein pre-fitted with a valve or blowpipe (31).

16. A resuscitation covering (11) as set forth in claim 2, which further includes a mouth-to-mouth resuscitation area (22) formed or to be formed with an opening.

17. A resuscitation covering (11) as set forth in claim 16, wherein the mouth-to-mouth resuscitation area (22) includes at least one predetermined breaking point (23) for a valve or blowpipe (31), or is fitted with a valve or blowpipe (31).

18. A resuscitation covering (41) as set forth in claim 2, which includes a recess (42) for placing the same against the neck of a resuscitated person.

19. A resuscitation covering (11, 41) as set forth in claim 2, wherein the emergency blanket (10) and/or the resuscitation covering (11) are/is at least partly transparent.

20. A resuscitation covering (11, 41) as set forth in claim 2, wherein the emergency blanket (10) and/or the resuscitation covering (11) comprise/comprises a sensor which is wired or based on wireless data transfer for the measurement of a person's vital function, for example heart rate, temperature, or ECG.
